(19) United States
(12) Patent Application Publication
(10) Pub. No.: US 2002/0065551 A1
(43) Pub. Date: May 30, 2002

(54) Method for immobilizing poly(HEMA) on stents

(76) Inventors: Levinas H. Koole, Gulpen (NL); Itzhak Binderman, Tel Aviv (IL)

Correspondence Address: Stanislaus Aksman, Esq., Hunton & Williams, Suite 1200, 1900 K Street, N.W., Washington, DC, 20006, US

(21) Appl. No.: 09/957,243

(22) Filed: Sep. 21, 2001

Related U.S. Application Data
(60) Provisional application No. 60/234,677, filed on Sep. 22, 2000.

(51) Int. Cl. B32B 1/08; B32B 1/02; B29D 22/00; A61L 2/00; B05D 3/00

(52) U.S. Cl. 623/1.25; 428/35.7; 428/36.91; 427/2.24

(57) ABSTRACT

The invention relates to the application of an adherent, biocompatible and stable polymeric coating onto the surface of stents with the aim to reduce the incidence of restenosis.

The invention provides for the application of hydrophilic polymeric coatings on surfaces, such as stent surfaces. The polymeric coating which is used is a methacrylate polymer, viz. a polymer obtained by polymerization of reactive methacrylate monomers. Suitable methacrylate polymers are those obtained by polymerization of 2-hydroxyethyl methacrylate (HEMA). Poly(HEMA) and other methacrylate polymers are polymeric biomaterials with known biocompatibility and proven biological safety.

### CROSS-REFERENCE TO RELATED APPLICATION

This application claims priority from U.S. provisional application, Ser. No. 60/234,677 filed Sep. 22, 2000, incorporated herein by reference.

### FIELD OF THE INVENTION

The invention is in the field of medicine as related to the use of metallic endovascular stents. More in particular, the invention relates to the application of an adherent, biocompatible and stable polymeric coating onto the surface of the stents with the aim to reduce the incidence of restenosis.

### BACKGROUND OF THE INVENTION

Endovascular stents find widespread use in clinical medicine, especially in interventional cardiology. A stent is normally used in conjunction with percutaneous translumenal angioplasty (PTA). This technique is applied to eliminate a so-called stenosis, a local obstruction of the blood flow in an artery, due to the presence of an atherosclerotic plaque. If the artery is a coronary artery, the procedure is known as PTCA or Dottering. During PT(C)A, a balloon is inserted into the stenosis, and the balloon is subsequently pressurized and inflated. The atherosclerotic plaque is then cracked and pushed radially into the blood vessel wall. Deflation of the balloon thereafter results in the desired re-canalization of the blood vessel. While PT(C)A is executed frequently, it is clear that there are two persisting major complications.

The first complication is known as elastic recoil. Clinical experience clearly revealed that the blood vessel wall, after PT(C)A, is usually instable. There is a clear risk for re-narrowing of the blood vessel wall due to a process which is known as elastic recoil.

The second complication is known as restenosis. Restenosis is a biological phenomenon, the backgrounds of which are currently well understood. In essence, restenosis is the response of the vessel wall structure to the PT(C)A procedure, which results in rupture of the medial and adventitial tissues that make up the outer parts of the blood vessel structure. Restenosis is due, in part, to intimal thickening, which occurs as vascular smooth muscle cells and inflammatory cells (primarily monocytes and macrophages) migrate and proliferate within the arterial media and intima. These cells produce abundant extracellular matrix. This further expands the intimal area, thereby impinging upon the lumen. Further, restenosis is due in part to arterial remodeling. This phenomenon occurs as the adventitia becomes rich in collagen which is produced by fibroblasts. This creates a “noose” around the artery thereby shrinking it further. Restenosis normally becomes manifest within 6 months after PT(C)A.

It has become clear in recent years that the implantation of a stent, immediately after the PT(C)A procedure, helps to reduce the incidence of both elastic recoil and restenosis. A stent is a tubular metallic scaffold in the form of a mesh that is expandable in radial directions. A stent is inserted in its narrow form, and guided to the site of the stenosis, immediately after PT(C)A. Once in position, the stent is expanded radially, usually again through the use of a balloon. Routinely, the stent is mounted in its narrow form on the deflated balloon, and guided as such toward the stenosis. Inflation of the balloon deploys the stent that is then pushed against the blood vessel wall. In this situation, the stent provides local mechanical support to the blood vessel. The requirement of the stent to be stable in a narrow form (prior to and during the introduction), and to be stable in a widened form (after the expansion) has resulted in the use of metals for their construction. The physical/mechanical properties of metals allow for a smooth elastic or plastic deformation from the narrow form toward the wide form. Examples of metals and alloys that are used in the manufacture of endovascular stents are, but are not limited to: stainless steel, tantalum, and Ni-Ti alloys with superelastic or shape-memory properties.

It is quite obvious that implantation of a stent is an effective strategy with respect to the prevention of elastic recoil. It has become clear however, that the risk for restenosis remains significant, regardless of both the construction of the stent, and the stent geometry.

Several points are noteworthy:

A variety of different stent materials have been developed and proposed during the last years, with the specific aim to reduce or even to eliminate the risk for restenosis.

A variety of different stent designs have been developed and proposed during recent years, with the specific aim to reduce or even to eliminate the risk for restenosis.

It has been suggested that the occurrence of restenosis in the presence of a stent is related to the fact that stents are not biocompatible. For instance, metals have a notoriously bad blood-compatibility; contact of blood with a metallic surface is known to lead to very fast coagulation. It has also become clear that the presence of the metallic stent inside the blood vessel structure may, by itself, provoke smooth muscle cells and other cells in the media and adventitia, to become engaged in a process of rapid proliferation resulting in restenosis.

It has also been suggested that the application of a polymeric coating onto the surface of the stent may result in a better biocompatibility, or (in other words) in a decreased propensity of the stent to induce enhanced proliferation of neighboring cells. It is important to realize that the coating does not influence the mechanical properties of the stent. Important features such as strength, expandability, etc., are essentially unimpaired). Investigations with a variety of coatings, however, have shown that coatings generally lead to enhanced inflammatory responses.

It has been suggested that such a coating should consist of a biocompatible biomaterial, preferably a polymer with hydrophilic properties. The coating can also act as a temporary depot for a drug, which is released locally from the surface of the stent, as the coating is in contact with water.

There is a clear need for a polymeric coating that does not compromise the biocompatibility of the bare untreated metallic stent, and which could serve as a platform for local delivery of a pharmacologically active agent, from the surface of the stent. Such a drug could be used, for example, for the inhibition of restenosis, or to prevent problems due to blood coagulation at the surface of the stent.

Adhering a hydrophilic polymeric coating onto a metallic substrate poses a technical problem. The coating will absorb water molecules upon immersion of the stent in water, or into a water-rich environment. The swollen coating will then tend to dissolve or at least to detach from the metallic surface. Desintegration of the polymeric coating from the surface of the stent is absolutely intolerable; detached polymer particles may act as emboli, and lead to obstructions in small blood vessel, e.g., in the coronary arterial system, or in the lungs or in the brain.

The present application is directed to a technical procedure for the application of hydrophilic polymeric coatings on stent surfaces. The polymeric coating which is used is a methacrylate polymer, viz. a polymer obtained by polymerization of reactive methacrylate monomers. Suitable methacrylate polymers are those obtained by polymerization of 2-hydroxyethyl methacrylate (HEMA). Poly(HEMA) and other methacrylate polymers are polymeric biomaterials with known biocompatibility and proven biological safety.

### RELATED ART

There are several patents covering coatings of endovascular stents and methods of manufacturing such coated stents. There is one patent which discloses the application of poly[(N-vinyllactam)/alkylmethacrylate] copolymer coatings on metallic wires, in such a way that the hydrophilic polymer is immobilized atop of a thin layer of polyethersulfone. A survey of the most relevant prior art is given below.

U.S. Pat. No. 6,086,547 (issued Jul. 11, 2000) discloses a wire for medical use, which is coated in a continuous extrusion-like procedure, first with a thin layer of poly(ethersulfone) and thereafter with a thin layer of a hydrophilic copolymer. Said copolymer is a hydrophilic copolymer built up of an N-vinyllactam (N-vinylpyrrolidinone is particularly suitable), and an n-alkylmethacrylate. Said copolymer was designed to render the wire both haemocompatible (non-thrombogenic) and lubricious.

U.S. Pat. No. 5,837,313 (issued Nov. 17, 1998) describes a method of making implantable open-lattice metallic stents. This approach uses a solvent mixture of uncured polymeric silicone material and a crosslinker. The coatings are cured in situ to form an adherent layer on the stent surface. Biologically active species can be incorporated into the coating, to be released after implantation.

U.S. Pat. No. 5,722,984 (issued Mar. 3, 1998) discloses a coating material with anti-thrombotic properties, while the coating contains embedded radioactive phosphorus-32. The antithrombotic coating consists of phosphorylcholine. Some of the phosphorus atoms in the phosphorylcholine headgroups are the phosphorus-32 isotope. The single coating is therefore both anti-thrombogenic and radioactive; radioactivity is claimed to suppress restenosis in response to PT(C)A trauma.

U.S. Pat. No. 5,871,437 (issued Feb. 16, 1999) discloses an endovascular stent coated with a biodegradable or a non-biodegradable polymeric coating (thickness<100 micrometer). The coating can contain a radio-active source of beta-emitting particles, with the aim to inhibit restenosis in response to PT(C)A trauma. The polymeric coating has anti-thrombogenic properties.

U.S. Pat. No. 05,059,166 (issued Oct. 22, 1991) discloses a concept of an intraarterial stent with an anti-thrombogenic coating, which contains a radio-isotope. The aim of the radio-isotope is to realize prophylaxis of restenosis by inhibiting the proliferation of cells in close proximity to the stent.

EP-A-0,993,308 (published Dec. 30, 1998) describes stents with a base body which are coated with a carrier polymer linked to perfluoroalkyl chains. These perfluoroalkyl chains protrude from the stent surface like a brush. This invention also encomapsses the method for producing such stents, and to use them in the prophylaxis of restenosis.

EP-A-0,832,655 (published Apr. 1, 1998) describes a coating and a method for implanting open-lattice metallic stent prostheses. The coating is biostable and consists of a thin polymeric layer which contains an amount of a biologically active material, particularly heparin.

### SUMMARY OF THE INVENTION

This invention is directed to methods for application of the biocompatible polymeric biomaterial poly(HEMA) and other methacrylate polymers as a coating for endovascular stents. Poly(HEMA) is the polymer that is derived from the monomer 2-hydroxy-ethylmethacrylate (HEMA); the structural formulas of HEMA and poly(HEMA) are schematically drawn in FIG. 1.

FIG. 1. Structural formulas of HEMA and poly(HEMA).

More specifically, this application describes a novel technique for application of poly(HEMA) or other methacrylate polymers, as a coating to the stent surface, in such a way that the coating is strongly adherent, i.e., there is no detachment of the coating from the underlying stent surface. While this application is focused on the use of poly(HEMA) as the coating biomaterial, it is specifically mentioned that the application technique is also applicable to other hydrophilic and non-hydrophilic polymeric coating materials. Suitable methacrylate polymers according to the invention are those obtained by polymerization of monomers selected from hydroxyethoxyethyl methacrylate (HEEMA), hydroxydiethoxyethyl methacrylate (HDEEMA), methoxyethyl methacrylate (MEMA), methoxyethoxyethyl methacrylate (MEEDA), metoxydiethoxyethyl methacrylate (MDEEMA), ethylene glycol dimethacrylate (EGDMA) and mixtures thereof. Preferably poly(HEMA) is used. These polymeric coatings can be crosslinked or non-crosslinked. These polymeric materials can be copolymers, terpolymers or even more complex macromolecular systems, or physical blends. It is also explicitly noted that other medical and non-medical devices and surfaces can be coated with hydrophilic and non-hydrophilic polymeric coatings, according to the poly(HEMA) or other methacrylate polymers, as described in this application. Other examples of application of poly(HEMA) or other methacrylate polymers coatings include, but are not limited to: food containers, beverage containers, other equipment that is in contact with food, and different medical equipments. It has been shown for stents with a poly(HEMA) coating that was applied according to a procedure that is described in this application, that favorable results are obtained in terms of occurrence of inflammation, thrombosis, and unfavorable tissue reactions.

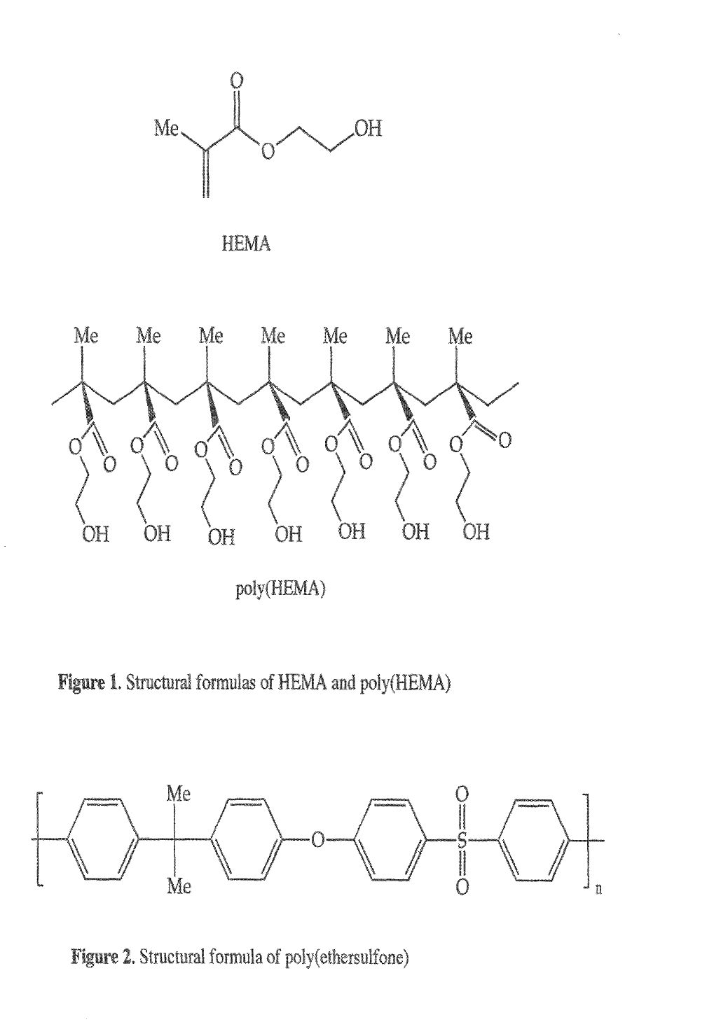

The essential point in this application is the method of adherence of the methacrylate polymer biomaterial to the metallic surface of the stent. It is essential in this application that a binder polymer is used; this binder polymer is preferably poly(ethersulfone), which is known to show strong adherence to metallic surfaces. The structural formula of poly(ethersulfone) is given schematically in FIG. 2 but it is explicitly mentioned that related polymeric structures which show excellent adherence to metallic surfaces, may also be used as binders in this specific application. The method of coating the stent with a methacrylate polymer, such as poly(HEMA), using this specific technology, is the subject of this patent application.

FIG. 2. Structural formula of poly(ethersulfone)

Stents, coated with poly(HEMA) according to the novel method that is described in this application, have been tested in a well-established animal model, i.e., in the iliac artery of New Zealand white rabbits. A published and GLP-approved experimental protocol was followed. The study comprised 8 animals and 16 stents (8 stents were coated with poly(HEMA), and 8 stents were untreated controls). The animals were sacrificed 28 days post-implantation. The most important findings of the study were:

There was no thrombus formation induced by the stent coating.

The presence of the coating had no impact on arterial size, neointimal growth, or medial thickness, i.e. not in an adverse manner and not in a beneficial manner. There was no evidence for added injury or induced recoil. The extent of luminal occlusion along the stent, in the proximal, middle, and distal segments, and the worst stenosis anywhere along the stent, were statistically identical between the coated stents and the untreated controls. These results are remarkable and exciting to anyone skilled in this specific art. Our poly(HEMA) coating—unlike a variety of other coatings—does not induce added inflammation, thrombosis and any other unfavorable tissue reactions. Such coatings can be useful for use in human patients. Possible applications are, but are not limited to: as a lubricious coating, or as a platform for local delivery of an anti-restenosis drug, or another pharmacologically active agent, from the stent surface into the vessel wall or into the circulation.

### DETAILED DESCRIPTION OF THE INVENTION

Poly(HEMA) and polyethersulfone can be purchased in pure form from commercial sources. Poly(HEMA) can also be prepared from purified HEMA monomer, for example in a radical polymerization reaction at elevated temperature. This chemistry is known to those skilled in the art.

Deposition of the poly(HEMA) coating is accomplished in two distinct steps. In the first step, the clean metallic stent is coated with a thin layer of the polymer poly(ethersulfone). In this first coating step, a solution of the polymer poly(ethersulfone) in a solvent, or in a solvent mixture, can be used. Examples of such a solvent are, but are not limited to: dimethylsulfoxide, N,N-dimethylformamide, N-methyl-2-pyrrollidinone. The solvent mixture can be prepared by combining these different solvents. The concentration of poly(ethersulfone) in the coating solution is preferably in the range 0.1-40% by mass. Application of the poly(ethersulfone) coating can be performed, for example, via a dip-coating procedure or via a spray-coating procedure. In the second step, the stent with the poly(ethersulfone) coating is coated with poly(HEMA). In this second step a solution of poly(HEMA) in a solvent, or in a solvent mixture, is used. Examples of such a solvent are, but are not limited to: dimethylsulfoxide, N,N-dimethylformamide, N-methyl-2-pyrrollidinone. The solvent mixture can be prepared by combining these different solvents. The concentration of poly(HEMA) in the coating solution is preferably in the range 0.1-40% by mass. Application of the poly(ethersulfone) coating can be performed, for example, via a dip-coating procedure or via a spray-coating procedure.

It is essential that the first and second coating steps are followed by: (i) a phase of 24 hours in which the coated stent is allowed to dry under ambient conditions (room temperature, relative humidity in the range 0-30%); (ii), a heat treatment. After the first step, heat treatment is essential to achieve complete evaporation of solvent residues. After the second coating step, heat treatment is essential in two different respects: (i) to achieve physical entanglement of the poly(ethersulfone) macromolecular chains and the poly(HEMA) macromolecular chains; (ii) to achieve complete evaporation of residual solvent molecules.

The heat treatment after the first step is, for example: heating of the coated stent at 120° C. in an inert atmosphere of purified nitrogen, for a period of 12 hours. The heat treatment after the second step is, for example: heating of the coated stent at 120° C. in an inert atmosphere of purified nitrogen, for a period of 24 hours.

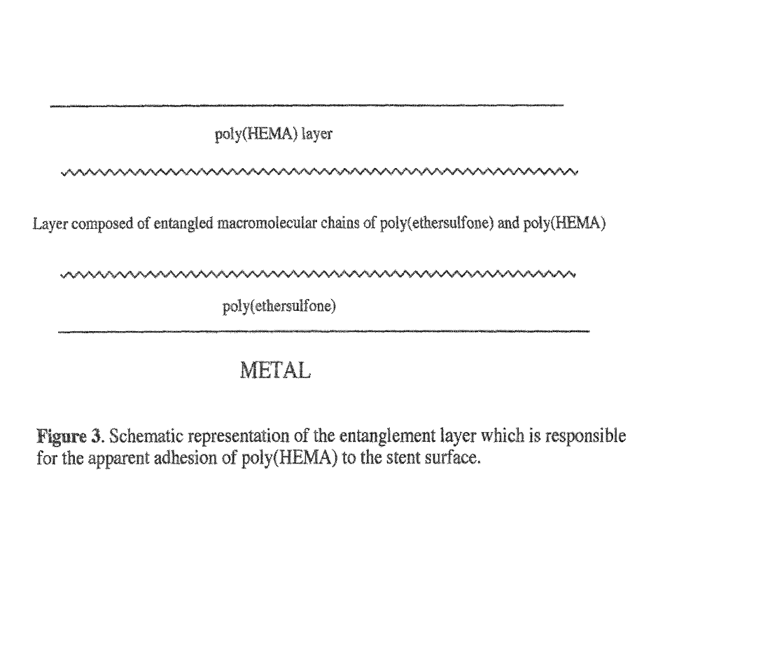

The physical entanglement that is achieved during the heating phase after the second coating step is crucial in this application. The physical entanglement results from the use of a solvent system for poly(HEMA), which is also capable of dissolving the outermost parts of the poly(ethersulfone) coating during the second coating step. The heat treatment after the second coating step further facilitates the physical entanglement. The entanglement is responsible for the observed strong adherence of the poly(HEMA) coating. The poly(HEMA) coating is capable of swelling and water-uptake upon immersion of the coated stent in water or in an aqueous environment, and the physical entanglement of the poly(HEMA) macromolecular chains in the underlying poly(ethersulfone) matrix prevents their dissolution. FIG. 3 further illustrates the principle of adherence of the poly(HEMA) coating via physical entanglement with the underlying polyethersulfone layer.

FIG. 3. Schematic representation of the entanglement layer which is responsible for the apparent adherence of poly(HEMA) to the stent surface.

In this way, a unique and novel principle for adhering hydrophilic polymeric materials to different materials (including metals) has been explored. As was mentioned above, this principle is applicable not only to poly(HEMA), but also to a variety of different hydrophilic and non-hydrophilic polymeric coating materials, and also to a variety of medical and non-medical devices and surfaces.

The coating is usually performed on stents in their native non-expanded state, but it is mentioned explicitly that an alternative option is to apply the coating in the expanded state of the stent; this latter option refers to so-called self-expanding stents in particular. The resulting coated stents were inspected thoroughly by means of optical and scanning electron microscopy. These techniques showed the presence of a uniform and smooth coating, both on the inner and on the outer surfaces of the stents. Importantly, it could be verified that the coating remains adherent and intact during and after expansion of the stent. The most difficult problem associated with both the dip-coating procedure, and the spray-coating procedure was to avoid deposition of coating between the struds of the stent. This proved to be possible, after precise optimization of essential process parameters such as: number of sprays or dips, concentration of the polymer in the solvent or solvent mixture. Subsequently, stents were sterilized by treatment with ethyleneoxide gas. There was no evidence for any degradation or any structural deterioration of the coating due to the sterilization. Although these results were obtained using poly(HEMA), similar results can be obtained using the other methacrylate polymer described herein-above.

### EXPERIMENT 1

Experimental protocol.

Sixteen stainless steel slotted-tube stents were tested in a well-established experimental model, which uses denuded iliac arteries of New Zealand white rabbits. Evaluation is based on state-of-the-art techniques for precise histological study of intact stented arteries and quantitative analysis of the inflammatory and proliferative cellular responses. The rabbits were treated orally with aspirin at a dose of 5 mg/kg/d beginning 1 day prior to stent placement. Anesthesia was obtained with ketamine and xylazine (intramuscular) and maintained as needed with intravascular Nembutal. Direct exposure of the femoral arteries was performed and a 3 French Fogarty balloon catheter was introduced through a surgical arteriotomy. The endothelium of the iliac artery was denuded when the catheter was inflated and withdrawn in its inflated state three times. Stents mounted on 3-mm PTCA balloons were deployed in the iliac artery proximal to the inguinal ligament. Balloon expansion was achieved using gradual steady increase in pressure to 8 atm. Pressure was maintained for 15 seconds, and released for an additional 30 seconds. Reproducibly precise stent placement was ensured by direct visualization and digital palpation of the artery, inguinal ligament, balloon, and stent. All animals received an intravenous bolus of heparin (100 U/kg) at the time of the first arteriotomy. Animals were housed in individual cages. After 28 days, the animals were euthanized with a lethal injection of intravenous Nembutal or Euthaniasia solution 5, and perfused clear with Ringer's lactate via left ventricular puncture. Arteries were surgically excised, fixed, end embedded in methacrylate. Histologic 5-mm sections were cut with a tungsten carbide knife, and stained with Verhoeffs tissue elastin stain, hematoxylin, and eosin. Morphometric analysis including the cross-sectional areas of neointima, media, and lumen and circumference of the stent, was performed using computer-based digitized image analysis at three sites along each stented segment (proximal end, middle, and distal end). All data are presented as mean ± SE. A two-tailed t-test was used for statistical analysis. This allowed for either positive or negative effects of coatings, with values of p<0.05 considered significant.

Results

Bilateral stent implantation was performed in 8 animals. Sixteen out of sixteen (100%) were inserted into the femoral arteriotomy without any difficulty. There were no differences seen upon histological examination of this stent 28 days after implantation. All animals survived until the planned time of sacrifice. At harvest, all stents were properly positioned in the mid-portion of the iliac artery. No wound infections were evident. Only two of the 16 stents were found to be occluded, despite the fact that only aspirin was used as chronic anticoagulant. There was one stent occluded in each group (i.e., one coated stent was occluded, and one uncoated stent was occluded). This corresponds to a thrombosis rate of 12.5%, which is in line with previous and comparable data which showed a thrombosis rate in the range 12-42%. It is noteworthy that the thrombosed coated stent had slipped off the balloon and was not positioned propertly. The positioning of the stent, rather than the material characteristics, likely caused the clotting, and we can likely discard this specimen from the calculations.

The arterial response 28 days after implantation of stainless steel stents consists of: (i), neointimal thickening and (ii), arterial remodeling.

Ad. (i): Intimal thickening occurs as vascular smooth muscle cells and inflammatory cells (primarily monocyte/macrophages) migrate into and proliferate within the arterial intima and media. These cells also produce abundant extracellular matrix that serves to further expand the intimal area within the bonds of the stent, thereby impinging upon the lumen. Intimal area grows over the first 14 days after stent implantation, and then remains relatively constant between 14 and 28 days. Examination of stainless steel stents with or without coatings showed all of these elements. A thick neointima developed separating the struts from the lumen. This consisted of smooth muscle cells and monocyte macrophages. Monocytes were also seen adherent to the luminal surface.

Ad. (ii): Arterial remodeling occurs as the adventitia becomes rich in collagen, produced by fibroblasts, in effect creating a “noose” around the artery and shrinking it further. This process is most evident between 14 and 28 days after stent implantation in experimental animals.

In a quantitative sense, the results of the animal experiments are compiled in Tables 1, 2, and 3.

TABLE 1
Intimal and medial response after 28 days.

| | Neointima | Media | Stent Radius | |
|---|---|---|---|---|
| Coating | (mm²) | (mm²) | (mm) | % Stenosis |
| None | 0.81 ± 0.08 | 0.18 ± 0.01 | 1.16 ± 0.03 | 32 ± 4 |
| Poly(HEMA) | 0.90 ± 0.05 | 0.25 ± 0.04 | 1.20 ± 0.02 | 29 ± 2 |

TABLE 2
Percentage of occlusion after 28 days.

| Coating | Proximal | Middle | Distal | Worst |
|---|---|---|---|---|
| None | 19.1 ± 2.7 | 19.2 ± 3.1 | 19.1 ± 1.3 | 23.4 ± 2.4 |
| Poly(HEMA) | 21.6 ± 3.6 | 18.8 ± 1.6 | 20.6 ± 2.4 | 24.9 ± 3.5 |

TABLE 3
Response of the lumen after 28 days.

| Coating | Average occlusion (%) | Average lumen (mm²) |
|---|---|---|
| None | 19.6 ± 2.3 | 3.36 ± 0.31 |
| Poly(HEMA) | 20.3 ± 1.6 | 3.16 ± 0.49 |

Conclusions

The experimental data lead to the following important and novel conclusions:

The poly(HEMA) stent coating, which is applied according to the novel method described in this application, and which essentially rests upon the formation of a polymeric entanglement layer for the adherence of the hydrophilic polymer poly(HEMA) to the stent struts, does not induce any thrombotic reaction.

The addition of the coating exerted no significant effect upon the arterial size, neointimal growth, or medial thickness in an adverse of beneficial manner. There was no evidence for added injury or induced recoil. The extent of luminal occlusion along the length of the stent, in the proximal, middle, and distal segments, and the worst stenosis anywhere along the stent, were statistically identical between the coated and uncoated stents.

The poly(HEMA) coating demonstrates no: (i) added inflammation, (ii), added thrombosis and (iii), tissue reaction, which is very remarkable and novel, since most polymeric coatings applied to stents are known to be associated with (i) and/or (ii) and/or (iii).

These data indicate that the subject poly(HEMA) coating can serve as a biocompatible coating for stents. Such a coating can be used as a lubricious coating, or as a platform for local drug delivery. Examples of such drug delivery strategies are, but are not limited to: (i) administration of an anti-restenosis drug from the surface of the poly(HEMA) coated stent into the bloodvessel wall, (ii), release of an anticoagulant agent from the surface of the poly(HEMA) coated stent into the circulation, (iii) release of a vasodilating drug from the surface of the poly(HEMA) coated stent.

Having now fully described the invention, it will be understood by those skilled in the art that the invention may be performed within a wide and equivalent range of conditions, parameters and the like, without affecting the spirit or scope of the invention or any embodiment thereof. It will also be understood that the specific examples are for the purpose of illustration only, and are not intended to be limiting, unless otherwise specified.

What is claimed is:

1. A method of providing a coating of a methacrylate polymer on a surface, said method comprising:
(a) applying a first coating of a first polymer on said surface, said first polymer featuring strong bonding to the said surface;
(b) heat treatment of the coated surface to ensure adequate binding of said first coating to the said surface;
(c) application of said methacrylate polymer coating, using a solvent or a solvent system, in which both methacrylate polymer and said first polymer are soluble;
(d) heat treatment in order to achieve the formation of an entanglement layer, whereby the immobilization of the methacrylate polymer is essentially accomplished.

2. The method of claim 1, wherein said methacrylate polymer is a polymer obtained by polymerization of monomers selected from 2-hydroxyethyl methacrylate (HEMA), hydroxyethoxyethyl methacrylate (HEEMA), hydroxydiethoxyethyl methacrylate (HDEEMA), methoxyethyl methacrylate (MEMA), methoxyethoxyethyl methacrylate (MEEDA), metoxydiethoxyethyl methacrylate (MDEEMA), ethylene glycol dimethacrylate (EGDMA) and mixtures thereof.

3. The method of claim 2, wherein said methacrylate polymer is poly(2-hydroxyethyl methacrylate).

4. The method according to claim 1, wherein said first polymer is polyethersulfone.

5. The method according to claim 1, wherein said surface is made of metallic, polymeric, or ceramic material.

6. The method according to claim 1, wherein step (c) is carried out by means of dipping, spraying or extrusion.

7. The method according to claim 1, wherein at least one releasable active ingredient is present in said coating.

8. The method according to claim 7, wherein said active ingredient is a substance with known pharmacological, antibacterial, or antifungal activity.

9. The method according to claim 7, wherein said active ingredient is a drug or a radioactive substance.

10. The method according to claim 9, wherein said drug is an anti-restenosis drug.

11. A medical device coated with the method according to claim 1.

12. A medical device according to claim 11, which is an endovascular stent.

13. A container, tubing and related equipment and devices which serve for preparation, transport, or storage of food, beverages and similar consumable products, coated with the method according to claim 1.
